(19) United States
(12) Patent Application Publication
(10) Pub. No.: US 2026/0144833 A1
(43) Pub. Date: May 28, 2026

(54) COMPOSITIONS AND METHODS USING A COMBINATION OF AT LEAST ONE FIBER AND AT LEAST ONE PROBIOTIC TO IMPROVE AND/OR MAINTAIN OPTIMAL CHOLESTEROL LEVELS

(71) Applicant: SOCIÉTÉ DES PRODUITS NESTLÉ S.A., Vevey (CH)

(72) Inventors: Marc GARCIA-GARCERA, Vucherens (CH); Sami DAMAK, Lausanne (CH); Bernard BERGER, Maracon (CH); Fabio MAINARDI, Lausanne (CH); Clara Lucia GARCIA-RODENAS, Forel (CH)

(21) Appl. No.: 19/123,515

(22) PCT Filed: Oct. 30, 2023
(86) PCT No.: PCT/EP2023/080205

§ 371 Date: Apr. 23, 2025

(30) Foreign Application Priority Data
Oct. 31, 2022 (EP) 22204656.7

(51) Int. Cl. A61K 35/747; A61K 31/702; A61K 31/717; A61K 31/732; A61K 31/736; A61K 35/745; A61K 36/55; A61K 36/736; A61P 3/06

(52) U.S. Cl. CPC A61K 35/747; A61K 31/702; A61K 31/717; A61K 31/732; A61K 31/736; A61K 35/745; A61K 36/55; A61K 36/736; A61P 3/06

(57) ABSTRACT

A composition contains a combination of at least one fiber and at least one probiotic and is formulated for administration to a subject in a condition of challenged gut microbiome such that the combination of at least one fiber and at least one probiotic improves and/or maintains optimal cholesterol levels in a subject in a condition of challenged gut microbiome. Preferably the subject is experiencing a microbiome stressor, such as one or more of a dietary stressor, antibiotic, other medications, infections, intense exercise, stress, alcohol, travel and combinations thereof; more preferably at least a dietary stressor; most preferably at least a high fat diet, such as a Western high fat diet or a ketogenic diet. The method can achieve at least one result that is improving and/or maintaining optimal cholesterol levels in a subject in a condition of challenged gut microbiome.

The present disclosure generally relates to compositions for use in improving and/or maintaining optimal cholesterol levels in a subject in a condition of challenged gut microbiome. The present disclosure further relates to methods of using and making such compositions.

### BACKGROUND OF THE INVENTION

The human gut microbiome is an ecosystem of trillions of bacteria, contributing to overall metabolism and health. Throughout life, the gut microbiome is challenged by one or more of unhealthy diets, antibiotics, other medications, infections, intense exercise, or alcohol.

Such challenges in the gut microbiome may lead to metabolic disturbances and have a negative impact on health overall.

At the same time, different factors, such as lack of exercise and physical activity, stress and hormones, obesity or overweight, smoking, alcohol, medications, medical conditions, unhealthy diets, such as high fat diets, may lead to increases in cholesterol levels in blood which may further cause cardiovascular and circulatory, endocrine and nervous system conditions and/or diseases.

Together with the challenged microbiome the increase of cholesterol levels may become even more drastic, and well-described methods and compositions may have no or insufficient effect for improving cholesterol to optimal levels, which has not been addressed previously.

Thus, there is a need to identify nutrients necessary to improve and/or maintain optimal cholesterol levels under conditions of challenged gut microbiome.

Further, there is a need to provide nutrient compositions and methods for improving and/or maintaining optimal cholesterol levels under conditions of challenged gut microbiome.

### SUMMARY OF THE INVENTION

The clinical study disclosed herein demonstrates that intake of a combination of a fiber blend and a probiotic mixture improves and/or maintains optimal cholesterol levels in subjects undergoing a challenge of a diet with high fat and low fiber and having as the consequence challenged gut microbiome. Accordingly, the present disclosure generally relates to novel compositions and methods to improve and/or maintain optimal cholesterol levels in a subject in a condition of challenged gut microbiome by using a combination of a fiber blend and a probiotic mixture.

Benefits from this improvement may include, for example, prevention and/or treatment of cardiovascular and circulatory, endocrine and nervous system conditions and/or diseases in subjects with challenged gut microbiome.

Additional features and advantages are described herein and will be apparent from the following Figures and Detailed Description.

### BRIEF DESCRIPTION OF DRAWINGS

FIG. 1 is a graph showing results from the experimental example disclosed herein, demonstrating reduced cholesterol levels when nutritional intervention is given.

FIG. 2 is a graph showing results from the experimental example disclosed herein, demonstrating lower frequency of subjects with suboptimal cholesterol levels when nutritional intervention is given.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a vitamin” or “the vitamin” encompass both an embodiment having a single vitamin and an embodiment having two or more vitamins.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified.

The terms “at least one of” and “and/or” used in the respective context of “at least one of X or Y” and “X and/or Y” should be interpreted as “X,” or “Y,” or “X and Y.” For example, “at least one of resistance or recovery” and “resistance and/or recovery” should be interpreted as “resistance,” or “recovery,” or “both resistance and recovery.”

Where used herein, the terms “example” and “such as,” particularly when followed by a listing of terms, are merely exemplary and illustrative and should not be deemed to be exclusive or comprehensive. As used herein, a condition “associated with” or “linked with” another condition means the conditions occur concurrently, preferably means that the conditions are caused by the same underlying condition, and most preferably means that one of the identified conditions is caused by the other identified condition.

“Prevention” includes reduction of risk, incidence and/or severity of a condition or disorder. The terms “treatment” and “treat” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

As used herein, a prophylactically or therapeutically “effective amount” is an amount that prevents a deficiency, treats a disease or medical condition in an individual, or, more generally, reduces symptoms, manages progression of the disease, or provides a nutritional, physiological, or medical benefit to the individual.

As used herein, the terms “food,” “food product” and “food composition” mean a product or composition that is intended for oral ingestion by a human or other mammal and comprises at least one nutrient for the human or other mammal.

“Nutritional compositions” and “nutritional products,” as used herein, include any number of food ingredients and possibly optional additional ingredients based on a functional need in the product and in full compliance with all applicable regulations. The optional ingredients may include, but are not limited to, conventional food additives, for example one or more, acidulants, additional thickeners, buffers or agents for pH adjustment, chelating agents, colorants, emulsifies, excipient, flavor agent, mineral, osmotic agents, a pharmaceutically acceptable carrier, preservatives, stabilizers, sugar, sweeteners, texturizers, and/or vitamins. The optional ingredients can be added in any suitable amount.

“Probiotic” means microbial cell preparations or components of microbial cells with a beneficial effect on the health or well-being of the host. (Salminen S, Ouwehand A. Benno Y. et al “Probiotics: how should they be defined” Trends Food Sci. Technol. 1999:10 107-10).

The term “unit dosage form,” as used herein, refers to physically discrete units suitable as unitary dosages for human and animal subjects, each unit containing a predetermined quantity of the composition disclosed herein in an amount sufficient to produce the desired effect, in association with a pharmaceutically acceptable diluent, carrier or vehicle. The specifications for the unit dosage form depend on the particular compounds employed, the effect to be achieved, and the pharmacodynamics associated with each compound in the host.

A “subject” or “individual” is a mammal, preferably a human.

### Embodiments

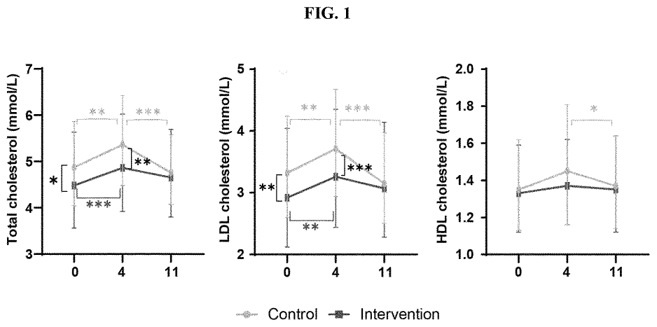

High fat diet has been shown to disturb the amount of lipids in the blood and to alter lipoprotein metabolism (dyslipidemia). The inventors investigated whether their short-term nutritional challenge induced dyslipidemia in the participants and if microbiota resilience was associated with plasma lipids. They measured the levels of cholesterol and triglycerides in plasma at baseline, after the nutritional challenge (day 4) and after the recovery period (day 11). They observed a significant increase in the levels of total and LDL cholesterol after the nutritional challenge (Wilcoxon rank test P<0.01, FIG. 1), which returned to baseline levels after the recovery period (P<0.001). The levels of HDL cholesterol did not change significantly with the challenge, but decreased after the recovery period (P=0.038). The levels of triglycerides, however, showed the opposite trend, with significant decreased values after the nutritional challenge (P=0.027) which were then recovered after the recovery period (P=7.2×10−4). Interestingly, the inventors observed that the LDL and total cholesterol changes were negatively correlated with the microbiome resistance (rho=−0.63, P=0.0035) but not the recovery (rho=0.02, P=0.87), suggesting that the less the microbiota resists to the nutritional challenge, the more the cholesterol levels increase in blood.

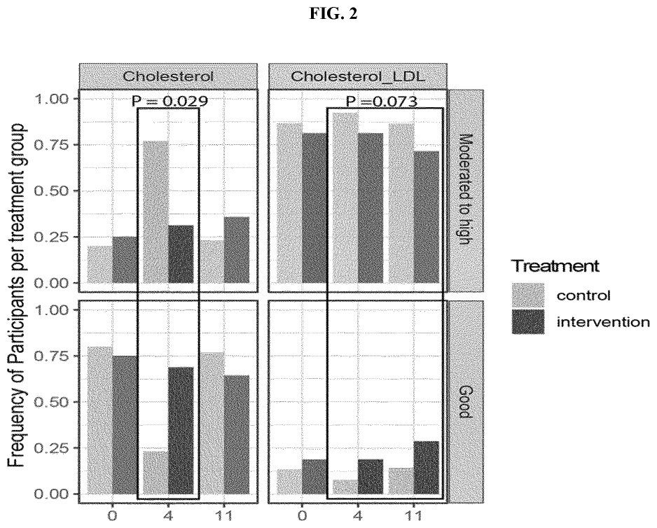

An important observation was that the levels of total and LDL cholesterol were significantly lower when participants were supplemented with the combination of probiotics and fibers (Wilcoxon test P<0.01). To gain insight in the potential benefit of the supplementation, the inventors categorized the participants based on whether their individual blood lipids levels were considered “optimal” or “suboptimal” and then assessed the evolution of the proportion of participants within each category before and after the high-fat diet as well as after the recovery period. During the control phase, 50% of the participants transiently transferred from the optimal to the suboptimal category for total cholesterol level after the high-fat challenge (FIG. 2). The reduction in the impact of the high fat diet on cholesterol levels was negatively correlated with the microbiome resilience (Spearman correlation rho=−0.53, P=0.0036). Similarly, the percentage of the participants that transferred from the optimal to the suboptimal category for LDL cholesterol was 10% during the control phase versus 0% during the intervention (Wilcoxon rank test P=0.073). Altogether this indicate that the increased microbiome resilience associated with probiotics and fibers supplementation could help reducing the acute dyslipidemia induced by a high-fat challenge. To explore potential mechanisms of action, the inventors hypothesized that the enhanced resilience could affect microbial cholesterol metabolism and impact the transport of cholesterol from the intestine to the bloodstream. They measured the differences in the levels of ApoB48, as a marker of lipid absorption, between before and after the high-fat challenge. They found that these differences were significantly smaller during the intervention (Wilcoxon rank test P=0.038). This suggests that the supplementation and associated microbial changes may have impacted the transport of cholesterol from the intestine to the bloodstream, leading to a maintenance of cholesterol level within the optimal range. Finally, the levels of ketone bodies, which increased after the high fat diet in the control population, did not change during the intervention (Wilcoxon test Pfdr>0.05) and were also significantly lower than the levels in the control phase (Pfdr<0.05, same test).

Accordingly, an aspect of the present disclosure is a composition for use in improving and/or maintaining optimal cholesterol levels in a subject in a condition of challenged gut microbiome, comprising a combination of at least one fiber and at least one probiotic in effective amounts.

In an embodiment, the composition is for use in preventing and/or treating of cardiovascular, circulatory, endocrine system and/or nervous system conditions and/or diseases.

In an embodiment, the challenged gut microbiome is caused by a microbiome stressor experienced by the subject.

In an embodiment, the microbiome stressor is selected from the group consisting of a dietary stressor; antibiotic; other medications; infections; intense exercise; stress; alcohol; travel; parenteral feeding; enteral feeding; short bowel syndrome; gut inflammation; chemotherapy; colon cancer; diarrhea; proton pump inhibitors; gluten-free diet; diet free of fermentable oligo-, di-, mono-saccharides and polyols (FODMAPs); and combinations thereof; preferably at least a dietary stressor; more preferably at least a high fat diet; most preferably a Western high fat low fiber diet or a ketogenic diet.

In a preferred embodiment, the stressor is a dietary stressor, such as a high fat diet, for example a Western diet or a ketogenic diet, or a low carbohydrate diet. As used herein, a high fat diet is a daily caloric intake in which greater than 35% of the daily caloric intake is from dietary fat, such as at least about 40% of the daily caloric intake from dietary fat, at least about 45% of the daily caloric intake from dietary fat, at least about 50% of the daily caloric intake from dietary fat, at least about 55% of the daily caloric intake from dietary fat, or at least about 60% of the daily caloric intake from dietary fat.

A Western diet is characterized by its highly processed and refined foods; high contents of sugars, salt, and fat; protein from red meat; and low content in fibers. “Low fiber” is considered as a diet with less than 15 g of fibers per 2000 calories per day.

As used herein, a low carbohydrate diet has no greater than about 15% of the daily caloric intake from carbohydrates, such as no greater than about 10% of the daily caloric intake from carbohydrates or no greater than about 5% of the daily caloric intake from carbohydrates.

In some embodiments, the subject has been consuming a high fat diet (e.g., a Western diet, or a ketogenic diet) or a low carbohydrate diet for at least one day (e.g., at least one week or at least one month) prior to a first administration of the combination of at least one fiber and at least one probiotic, with optional subsequent administrations of the combination of at least one fiber and at least one probiotic (e.g., daily administration over a time period of at least one week or at least one month).

In some embodiments, the subject consumes one of the compositions disclosed herein on a daily basis, for example each day for at least one week prior to the stressor or even at least one month prior to the stressor.

Yet another aspect is a unit dosage form of a composition comprising a combination of at least one fiber and at least one probiotic, the unit dosage form comprising an amount of the combination (of at least one fiber and at least one probiotic) effective to improve and/or maintain optimal cholesterol levels in a subject in a condition of challenged gut microbiome to whom the unit dosage form is administered.

In the embodiments disclosed herein, each of the at least one fiber is edible, meaning that all of the components of the fibers are safe and suitable for consumption by humans and/or animals. The at least one fiber comprises insoluble fiber and/or soluble fiber, preferably a blend of insoluble fiber and soluble fiber. In some embodiments, the at least one fiber can be selected from the group consisting of xylooligosaccharides, flax seed, partially hydrolyzed guar gum (PHGG), glucomannan, cellulose, prune powder, pectin such as apple peel pectin, and mixtures thereof. In some embodiments, the at least one fiber is at least two fibers, such as two, three, four, five, six or seven fibers and optionally more. Optionally one or more of Luo Han Guo fruit powder, xylitol or magnesium (e.g., magnesium citrate) can be included with the at least one fiber.

In some embodiments, the at least one probiotic can be selected from the group consisting of Lactobacillus acidophilus, Bifidobacterium lactis, Lactobacillus rhamnosus, Bifidobacterium longum, Lactobacillus plantarum, Bifidobacterium bifidum, Lactobacillus gasseri, and mixtures thereof. In some particular embodiments, the at least one probiotic can be a strain selected from the group consisting of Lactobacillus acidophilus La-14, Bifidobacterium lactis BI-04, Lactobacillus rhamnosus GG, Bifidobacterium longum BL-05, Lactobacillus plantarum Lp-115, Bifidobacterium bifidum Bb-06, Lactobacillus gasseri Lg-36, and mixtures thereof. In some embodiments, the at least one probiotic is at least two probiotic strains, such as two, three, four, five, six or seven probiotic strains and optionally more.

Another aspect is a method to improve and/or maintain optimal cholesterol levels in a subject in a condition of challenged gut microbiome, the method comprising administering, to the subject, the composition as described above.

In an embodiment, the present disclosure is a method of achieving at least one result selected from prevention and/or treatment of cardiovascular, circulatory, endocrine system and/or nervous system conditions and/or diseases, the method comprising improving and/or maintaining optimal cholesterol levels in a subject in a condition of challenged gut microbiome by administering, to the subject, the composition as described herein.

The at least one fiber may be administered to the individual as a total daily dose of about 5-40 g, preferably about 15-25 g. The at least one fiber may be administered in a composition comprising between about 300-1000 mg total fiber/g of dry composition.

The at least one probiotic may be administered to the individual as a daily dose of 1×10³ to 1×10¹², preferably 1×10⁷ to 1×10¹¹ cfu (cfu=colony forming unit). The at least one probiotic may be administered in a composition comprising between 1×10³ to 1×10¹² cfu/g of dry composition. The at least one probiotic may be alive, fragmented, or in the form of fermentation products (e.g., supernatant) or metabolites, or a mixture of any or all of these states.

The combination of at least one fiber and at least one probiotic is preferably orally administered in a composition such as a food composition.

The subject to whom the combination of at least one fiber and at least one probiotic is administered can be selected from the group consisting of a human infant, a human child, a human adolescent, a human adult and an elderly human.

The combination of at least one fiber and at least one probiotic can be administered to the individual by at least one route selected from the group consisting of oral, topical, enteral and parenteral. For example, the combination of at least one fiber and at least one probiotic can be administered in a composition selected from the group consisting of a nutritionally complete product, a drink, a dietary supplement, a meal replacement, a food additive, a supplement to a food product, a powder for dissolution, an enteral nutrition product, an infant formula, a capsule, and combinations thereof.

Optionally the combination of at least one fiber and at least one probiotic is administered in a composition further comprising at least one component selected from the group consisting of an amino acid, a protein, a nucleotide, a fish oil, a non-marine source of omega-3 fatty acids, a phytonutrient, an antioxidant, and mixtures thereof.

Another aspect is a method of making a composition to improve and/or maintain optimal cholesterol levels in a subject in a condition of challenged gut microbiome to whom the composition is administered, the method comprising adding at least one fiber to at least one probiotic and optionally at least one additional component.

The composition may be a food product, an animal food product, or a pharmaceutical composition. For example, the product may be a nutritional composition, a nutraceutical, a drink, a food additive or a medicament. A food additive or a medicament may be in the form of tablets, capsules, pastilles, a liquid, or a powder in a sachet, for example.

In some embodiments, the at least one probiotic is concurrently administered in a composition separate from the at least one fiber, for example in separate compositions administered to the same individual within one hour of each other, preferably within thirty minutes of each other, more preferably within ten minutes of each other, most preferably within one minute of each other.

The composition comprising the combination of at least one fiber and at least one probiotic is preferably selected from the group consisting of milk powder based products; instant drinks; ready-to-drink formulations; nutritional powders; nutritional liquids; milk-based products, in particular yoghurts or ice cream; cereal products; beverages; water; coffee; cappuccino; malt drinks; chocolate flavoured drinks; culinary products; soups; tablets; and/or syrups.

The composition may optionally comprise any milk obtainable from animal or plant sources, such as one or more of cow's milk, human milk, sheep milk, goat milk, horse milk, camel milk, rice milk or soy milk. Additionally or alternatively, milk-derived protein fractions or colostrum may be used.

The composition comprising the combination of at least one fiber and at least one probiotic may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials.

The composition comprising the combination of at least one fiber and at least one probiotic may also contain conventional pharmaceutical additives and adjuvants, excipients and diluents, including, but not limited to, water, gelatine of any origin, vegetable gums, ligninsulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like. Further, the composition may contain an organic or inorganic carrier material suitable for oral or enteral administration as well as vitamins, minerals trace elements and other micronutrients in accordance with the recommendations of Government bodies such as the USRDA.

The composition comprising the combination of at least one fiber and at least one probiotic may optionally contain one or more amino acids, a protein source, a carbohydrate source and/or a lipid source, particularly in embodiments of the composition that are a food product.

Any suitable dietary protein may be used, for example animal proteins (such as milk proteins, meat proteins and egg proteins); vegetable proteins (such as soy protein, wheat protein, rice protein, and pea protein); mixtures of free amino acids; or combinations thereof. Milk proteins such as casein and whey, and soy proteins are particularly preferred.

The composition comprising the combination of at least one fiber and at least one probiotic may be administered to humans or animals, in particular companion animals, pets or livestock. It has beneficial effects for any age group. Preferably, the composition is formulated for administration to infants, juveniles, adults or elderly. In some embodiments, the composition can be administered to mothers during pregnancy and lactation to treat the infant.

The composition comprising the combination of at least one fiber and at least one probiotic can be administered at least one day per week, preferably at least two days per week, more preferably at least three or four days per week (e.g., every other day), most preferably at least five days per week, six days per week, or seven days per week. The time period of administration can be at least one week, preferably at least one month, more preferably at least two months, most preferably at least three months, for example at least four months. In an embodiment, dosing is at least daily; for example, a subject may receive one or more doses daily. In some embodiments, the administration continues for the remaining life of the individual. In other embodiments, the administration occurs until no detectable symptoms of the medical condition remain. In specific embodiments, the administration occurs until a detectable improvement of at least one symptom occurs and, in further cases, continues to remain ameliorated.

### Example

The following non-limiting example generally illustrates the concepts underlying the embodiments disclosed herein.

Materials and Methods The study was based on a randomized, controlled, open, 2×2 crossover design. Twenty male and female, 18-45 years old eligible, participants were randomized to one of the 2 following sequences: 1) Control (no resilience enhancement) to resilience enhancement; 2) Resilience enhancement to control.

Each participant, irrespective of their cohort, followed the following study design:
Participants were followed for 21 days. During the first 10 days, diet was monitored but not controlled. After 10 days (beginning on Day 0), all participants also received for 5 days a dietary challenge consisting of a 60% fat, 15% carbohydrates, 25% Protein and 10 g of Fiber. After the dietary disturbance period, participants returned to their routine diet, which was monitored daily. For the participants enduring the intervention period, the combination of fibers and probiotics was administered for 22 days. Stool and plasma samples were collected throughout the study in different schedules. Moreover, food and comfort questionnaires were collected throughout the study. The trial was registered in clinical trials.gov under the number NCT04424329.

The challenge diet was designed to provide 60% fat, 25% proteins, 15% carbohydrates of the total daily caloric intake and 10 g of fibers per 2000 calories. Three different meal plans were created, two of which were repeated over the 5-day challenge period. The same meal plans were provided in the same order in both study phases.

The “diet builder” nutrition software (http://dnh-portal.dor.ch.nestle.com/) was used to design the diet challenge. The diet builder allows to choose one or more Food Composition Databases (FCDB) from which to select foods and to create meals by eating occasions (e.g. breakfast, lunch, etc) and determine the total daily nutritional composition (e.g. kcals, grams of fat, grams of protein, etc).

The estimated energy requirement (EER) of each study participant was calculated using the Institute of Medicine (IOM) equation. The “diet builder” nutrition software estimates the EER using IOM equation when inserting the demographic characteristics of the subjects including their level of self-reported physical activity in the tool. Based on the EER estimations, participants were grouped in four different clusters of energy requirements according to their different energy needs (2000, 2500, 3000 and 3750 kcal/day).

The food was prepared and given to the subjects as take away. They were instructed when possible to consume it all and to not eat additional items.

Before and after the diet challenge, participants were asked to record their food intake daily using an application developed in house (Nestlé Research app ref). During the diet challenge they were asked to record the amount of challenge diet consumed and any additional items they may have eaten. The data entered were converted by the application into nutrition information.

The inventors used a combination of probiotics and fibers as an interventional product. For probiotics, participants ingested one capsule containing 5×109 CFUs of a combination of Bifidobacterium Longum, Bifidobacterium Bifidum, Lactobacillus Gasseri), Lactobacillus Rhamnosus, Lactobacillus Plantarum, Lactobacillus acidophilus and Bifidobacterium lactis. For fibers, participants included 14 g of a powder containing a containing xylooligosaccharides, apple pectin, partially hydrolyzed guar gum, glucomannan, flax powder, cellulose and prune powder (For the proportions of each probiotic and fiber, see supplementary table 01). Both combinations are currently being commercialized by Pure Encapsulations, USA.

200 ug of stool were used for DNA extraction. 100 ng of DNA were sequenced using Illumina HiSeq 3000, to achieve 10 Gb of data. Raw data was then processed to assess the quality using FastQC, and annotated using the Atlas pipeline (Kieser, BMC Bioinformatics, 2020). Taxonomic annotation was used to characterize differences within and between participants using measurements of Alpha, Beta & gamma diversity. Community structure was built using taxonomic co-variance. Functional variability was extracted from gene annotation using RefSeq, KEGG, and eggNOG.

The study was carried out by Metabolon (Morrisville, North Carolina, USA) using an in-house HD4 Platform. In brief, Samples were extracted with methanol to precipitate the proteins and dissociate the small molecules bound to protein. The resulting extract was divided into five fractions: two for analysis by two separate reverse phase (RP)/UPLC-MS/MS methods using positive ion mode electrospray ionization (ESI), one for analysis by RP/UPLC-MS/MS using negative ion mode ESI, one for analysis by HILIC/UPLC-MS/MS using negative ion mode ESI, and one reserved for backup. Matabolites were identified using a proprietary software to match ions to a metabolon library of standards. All methods were subjected to different quality controls, including technical replication, negative (water and solvent) and known controls (cocktail of known metabolites, a list of the QC standards is given in supplementary table 02). Metabolites were quantified by peak area integration. Identified peaks were characterized by mapping against an in house reference library of metabolites Two-way Repeated Measures ANOVA and Principal Component Analysis (PCA) were used to analyze the data. For all analyses, missing values, if any, were imputed with the observed minimum for that particular compound. The statistical analyses were performed on natural log-transformed data. Data curation was applied when necessary to remove system artifacts, mis-assignments, redundancies and background noises.

Fasting EDTA plasma samples for metabolomics analysis were collected from 20 subjects at three time points (Day 0, Day 4, and Day 11) of the two phases, corresponding to the beginning, end and post-challenge event of both arms of the study.

55 μL of the plasma samples were centrifuged (1.5 min, 21130 rcf, 4° C.) and distributed into 2 aliquots of 20 μL. The remainder was used for a pooled sample. Aliquots of 5, 10, 15, 20 and 25 μL were derived from the pool as quality controls. All aliquots were kept at −80° C. until extraction. Prior to extraction, the samples were thawed on ice and 500 μL extraction solution (40:40:20 ACN:MeOH:H2O) containing isotopically labelled internal standards was added. After mixing on a vortex, the extract was centrifuged for 10 min at 21130 rcf at 4° C. The supernatant was dried overnight in a vacuum centrifuge at 4° C. and 5 mbar. The dried samples were resuspended in 50 μL 70% (v/v) acetonitrile in water and centrifuged for 1 min at 21130 rcf at room temperature. The resulting supernatants were transferred into glass vials for liquid chromatography coupled mass spectrometry (LC-MS) analysis.

Three μL of the samples were injected and separated on a Vanquish UHPLC (Thermo Scientific) with a hydrophilic liquid chromatography (HILIC) ZIC-PHILIC column (Merck Sequant) carrying the dimensions 100×2.1 mm, 5 μm with a ZIC-pHilic guard column (20×2.1 mm, 5 μm, Merck Sequant). The separation was achieved by applying a linear solvent gradient in normal mode at 0.2 mL/min flow rate and 35° C. of temperature. As mobile phase, solvent A was H2O with 10 mM ammonium acetate (NH4Ac) and 0.04% (v/v) ammonium hydroxide (NH4OH), pH˜9.3, and solvent B was acetonitrile (can). The gradient started with 90 solvent B was increased to initial conditions within 0.1 min and the column was equilibrated for 8.9 min, with a total analysis time of 30 min.

The eluting metabolites were analyzed with an Orbitrap Fusion Lumos mass spectrometer (Thermo Scientific) with a heated electrospray ionization (H-ESI) source with spray voltages of 3500 V and 3000 V for positive and negative mode, respectively. The sheath gas was 20 AU, and the auxiliary gas was kept at 15 AU. The temperature of vaporizer was 280° C. and the temperature of the ion transfer tube was 310° C. The full scan was measured with on-the-fly alternating positive and negative mode scans in four mass windows, which covered m/z ranges from 73 to 300 and from 195 to 1000. The resolution of the lower mass windows was set to 50'000 and the resolution of the upper windows was set to 60'000. Instrument control was performed with the Xcalibur software (Thermo Scientific).

An automated R pipeline consisting of a wrap-up of R packages (AlpsLCMS-version 0.0.0.9009)1 was applied for the pre-processing, peak-picking, feature reduction and putative annotation of the metabolomics data. An approach based on signal linearity of the pooled QC samples for the normalization and semi-quantification of the data was applied. Specifically, the metabolic features of five amounts of plasma (5 μl, 10 μl, 15 μl, 20 μl, and 25 μl, n=4) were normalized to each internal standard and a correlation analysis was performed. The internal standard that resulted in the best correlation between normalized feature and plasma amount was selected for later normalization. Features, where normalization did not result in a linear response after the correlation analysis (cutoff of r2>0.8) were excluded from further data analysis. After filtering for a CV of less than 30% in the 20 μl QCs, the data were manually cleaned for isotopes based on the mass difference and retention time of the features and redundant annotations from positive and negative mode (i.e. if a compound gave a signal in both modes) were removed.

Uni-and multi-variate statistical analyses were performed in the statistical software R (version 3.6.2). A principal component analysis (PCA) and heatmap were performed on the data for exploratory purposes. To explore the differences among visits, a baseline adjustment of each metabolite was applied for all subjects by dividing the corresponding metabolite values at Day 0 (pre-challenge). After discarding the normality assumption, the Kruskal-Wallis test for independent samples was employed between groups (control and nutritional intervention) at each visit to assess the statistical significances in a univariate manner. The Benjamini-Hochberg3 procedure was carried out on p-values to control the false discovery rate (FDR) in all comparisons. An FDR-corrected p-value of <0.05 was considered statistically significant.

Feces samples were homogenized with a solution of ortho-phosphoric acid containing S-labelled internal standards (acetate-D3, propionate-D5, isobutyrate-D7, butyrate-D5, isovalerate-D9 and valerate-D9) before being centrifuged at 2000 g to obtain fecal water. The fecal water samples were deproteinized with 5-sulfo-salicylic acid and short chain fatty acids (SCFAs) and branched chain fatty (BCFAs) were extracted with chloroform followed by a derivatization with tert-butyl-dimethylsilyl-imidazole (TBDMSIM). (REF) The analysis of SCFAs and BCFAs was performed by gas chromatography coupled with a mass spectrometer (GC-MS, Agilent Technologies, 6890 series XL MSD 5975 C). Chromatographic separation was performed on a DB-5MS (J&W Scientific, Folsom, CA.) with a runtime of 15 min. The mass spectrometer was operated in selected ion monitoring (SIM) mode. Quantification was calculated by comparison of the peak area ratio (not labelled/labelled) with the corresponding calibration curve. The following SCFAs were analyzed: acetic acid (C2:0), propionic acid (C3:0), butyric acid (C4:0), valeric acid (C5:0). And the BCFAs measured were isobutyric acid (iC 4:0), isovaleric acid (iC5:0), and 2-methyl-butyric acid (2-methyl C4:0) (quantified with isovalerate-D9 as internal standard). The SCFAs and BCFAs were expressed as μmol/g of wet feces. Values below the lower limit of quantification (LLOQ) were imputed by half of the LLOQ.

Stool consistency was assessed by the participants using the Bristol scale (ref) which ranks the stool from 1 (hard lumps) to 7 (watery) for every stool produced. Stool frequency was also recorded by the participants. Both were recorded daily throughout the study.

Blood was collected on days 0, 4 and 11. Serum samples obtained by blood clotting and spinning at 1500 g, 10 min, 4° C., were immediately frozen at −80° C. and send to MLM Medical Labs GmbH (Monchengladbach, Germany) in dry ice for permeability marker assessment. Intestinal fatty acid binding protein and IgM anti-endotoxin core antibody were analyzed by ELISA, using respectively the Human iFABP (KBH1541) and Human EndoCAb® IgM (HK504-IGM) ELISA kits from HycultBiotech (Beutelsbach, Germany) according to the manufacturer's instructions.

Fresh blood samples were collected into BD Vacutainer plasma tubes (VWR BDAM368495) at days 0, 4 and 11 either before breakfast (fasted state) or 5 h after high fat diet meal (postprandial state). Plasma was isolated by centrifugation of blood at 2000 g for 10 minutes at 4° C. and used for subsequent measurements of hsCRP, IL-16, IL-6, IL-8 and TNFα. Plasma hsCRP levels were measured using a commercial kit (Abbott Laboratories) and analyzer (Architect C8000; Abbott Laboratories). For cytokine analyses, plasma samples were diluted 1:2 and analyzed using a Mesoscale Custom V-PLEX human cytokine assay (Meso Scale Discovery (MSD), Inc., K151AOH-2) for IL-16, IL-6, IL-8 and TNFα according to the manufacturer's instructions. Briefly, samples were incubated on MSD plates for 2 h at room temperature with shaking. Plates were washed and incubated an additional 2 hours with detection antibodies. After washing, samples were analyzed in duplicates using the MSD Workbench software and treatment differences over days of intervention and fasting status are indicated (log-scale).

Gut comfort was assessed by visual analog scale from 0 to 10, 0 being no symptoms, and 10 the worst possible symptoms. Participants recorded the level of nausea, vomiting, audible abdominal sounds, abdominal cramps and flatulence, daily throughout the study.

All analyses were performed in R. All statistical tests used were two-sided, unless specified otherwise. Microbiome data was analyzed using genome-based mapping aggregates and defined at species level. Unless stated otherwise, microbiome data was assessed using non-parametric tests (Wilcoxon rank test and Mann-Whitney). Multiple testing was performed with the Benjamini-Hochberg procedure (FDR) whenever applicable (q-values). For fecal metabolites natural log-transformed data were used to perform statistical analyses.

### Results

FIG. 1 shows change in levels of plasma cholesterol over time. X-axis represents time (in days). Y-axis represents levels of the biomarkers. Colours represent the interventional state of the population, as described in the bottom legend. Significancy is defined by paired two-sided Wilcoxon rank test (*=Pfdr<0.05; **=Pfdr<0.01; ***=Pfdr<0.005). Panel A) shows the levels of total cholesterol in plasma; Panel B) shows the levels of LDL-cholesterol in plasma; Panel C) shows the levels of HDL cholesterol in plasma.

As it is shown in FIG. 1, the levels of total and LDL cholesterol were significantly lower when participants were supplemented with the composition as claimed.

FIG. 2 shows that the frequency of subjects with suboptimal cholesterol levels becomes lower when participants were supplemented with the composition as claimed.

This rigorous randomized controlled clinical study demonstrates that intake of a combination of a fiber blend and a probiotic mixture improve and/or maintain optimal cholesterol levels in a subject in a condition of challenged gut microbiome.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method of improving and/or maintaining optimal cholesterol levels in a subject in a condition of challenged gut microbiome, the method comprising administering a combination of at least one fiber and at least one probiotic in effective amounts.

2. The method of claim 1, wherein the challenged gut microbiome is caused by a microbiome stressor experienced by the subject.

3. The method of claim 2, wherein the microbiome stressor is selected from the group consisting of a dietary stressor; antibiotic; other medications; infections; intense exercise; stress; alcohol; travel; parenteral feeding; enteral feeding; short bowel syndrome; gut inflammation; chemotherapy; colon cancer; diarrhea; proton pump inhibitors; gluten-free diet; diet free of fermentable oligo-, di-, mono-saccharides and polyols (FODMAPs); and combinations thereof.

4. The method of claim 1, wherein the at least one fiber is selected from the group consisting of insoluble fiber, soluble fiber, and a mixture thereof.

5. The method of claim 1, wherein the at least one fiber is selected from the group consisting of xylo-oligosaccharides, flax seed, partially hydrolyzed guar gum (PHGG), glucomannan, cellulose, prune powder, pectin and mixtures thereof.

6. The method of claim 1, wherein the at least one fiber comprises at least two fibers.

7. The method of claim 1, wherein the at least one probiotic is selected from the group consisting of Lactobacillus acidophilus, Bifidobacterium lactis, Lactobacillus rhamnosus, Bifidobacterium longum, Lactobacillus plantarum, Bifidobacterium bifidum, Lactobacillus gasseri, and mixtures thereof.

8. The method of claim 1, wherein the at least one probiotic comprises a strain selected from the group consisting of Lactobacillus acidophilus La-14, Bifidobacterium lactis BI-04, Lactobacillus rhamnosus GG, Bifidobacterium longum BL-05, Lactobacillus plantarum Lp-115, Bifidobacterium bifidum Bb-06, Lactobacillus gasseri Lg-36, and mixtures thereof.

9. The method of claim 1, wherein the at least one probiotic comprises at least two probiotic strains.

10. Themethod of claim 1, wherein the combination of the at least one fiber and the at least one probiotic is orally administered to the subject.

11. The method of claim 1, wherein the subject is selected from the group consisting of a human infant, a human child, a human adolescent, a human adult, an elderly human, and a companion animal.

12. (canceled)

13. (canceled)

14. A method of achieving at least one result selected from prevention and/or treatment of cardiovascular, circulatory, endocrine system and/or nervous system conditions and/or diseases, the method comprising improving and/or maintaining optimal cholesterol levels in a subject in a condition of challenged gut microbiome by administering, to the subject, a combination of at least one fiber and at least one probiotic in effective amounts.

15. A method of making a composition to improve and/or maintain optimal cholesterol levels in a subject in a condition of challenged gut microbiome to whom the composition is administered, the method comprising adding at least one fiber to at least one probiotic.

16. A unit dosage form of a composition comprising a combination of at least one fiber and at least one probiotic, the unit dosage form comprising an amount of the combination of the at least one fiber and the at least one probiotic effective to improve and/or maintain optimal cholesterol levels in a subject in a condition of challenged gut microbiome to whom the unit dosage form is administered.

17. The method of claim 2, wherein the microbiome stressor is at least a dietary stressor.

18. The method of claim 2, wherein the microbiome stressor is at least a high fat diet.

19. The method of claim 2, wherein the microbiome stressor is a Western high fat low fiber diet.

20. The method of claim 2, wherein the microbiome stressor is a ketogenic diet.

21. The method of claim 1, wherein the at least one fiber comprises a mixture of insoluble fiber and soluble fiber.
